SEQUENCE LISTING (21 sequences)

SEQ ID NO: 1 - DNA, 1923 bp, Bos taurus
  source 1..1923: mol_type = genomic DNA
ggtaggacacgcaggcctccgccagggcagcacagacactgtgagagatgcttcaaccgtcactgccacatgcctgtggagcccagtgtctcttgcccggtgataaactgccacctgtcctgcggtgctaccttccacatgtgcaaagaggctgagcacgagctactctgccctttagagcaggttccatgcctcaactctgaatatggctgccccctcttcatgtcccgccacaagctggccaagcatctgcaagtgtgtcctgccagcgtggtctgctgctccatggagtggaaccgttggccaaacgtggactctgaaacaacgcttcatgagaacatcatgaaagagacccccgatgaggaatgcttggacacagccctggcccttcaggaccagaaggtcctttttaggtctctgaaaatggtagaacttttcccagaaactagagaacccacggaagaggaacctcctatgaatggtgaagccagctgggaggaaatggatggagcggtgggtggggaggatgctggtttggtaacaggctgctctctgtcagccatgaatggggaggtggtagagctaagtcaagaagaacgagaggcgttagccaaaaccaaagaagggatggacctggccaagtttgacaagtgggaaaatatgttcagcaaagagcatgcggcctcagctttaacaagctcatcagccagcagtgacagcaaaagcaggaacggcccagagaaagaacagatttccaacagcaatagaatggtagaggagggagctgccaaaggaaaagaaccacaggaagaccagaagcagcaggactttcatgcagccatagaaaagacaggacttgccccttggcaggatggtgttctggaaagactgaaaacagctgtggatgcaaaggactataatatgtacctggtgcacaatgggaggatgcttattcactttggtcagatgcctgcttgtacacccaaagagagagactttgtttacggcaacctggaggctcaggaagtgaagactgtttacaccttcaaagttccagtgagctactgtgggaagcgggctcgcctaggtgatgccatgagttgtaggccaagtgagcacaaagccgttgatacttcagatttagggatcactgtggaggacctgcccaagtcagatctcatcaagaccaccctcctgtgtgctttggaaagagaactcaaaggtcacgtcatctccgagtccaggagcattgacgggctgttcatggatttcgctacacagacatacagttttgagccagaacagttttcttctgggacggtgctggccgacctcttaaccactgccaacccaaacagcaatgcgaatgggctccacgtggagctccacagtgagtgtgtgaccaggagacacaacaagagcagctctgcctttactttcacctgcaacaaattcttcaggagggatgaatttcccctacatttcaagaatgttcacacagacattcagtcatgtctcaacggctggttccaacaccgatgccccctggcctacttgggatgtacctttattcaaaaccatttccgtcccccagggcaaaaggcaaaagtgatctacagtcaggagctcaagacctttgccatcaagccggaggttgcttcagagctgagtgagggaaggaagaacaaccatttctcaggtcgtgaaggaaaaaaccagaattctctaaccagcctgcccctggaggtcctgcagtacattgctgggttcctagacagcatcagcctgtcccagctctcccaggtgtccgtgctgatgaggagtatctgtgccactttgttacaagagagagggatggtcctcctgcagtggaagaagaagaggtattcccatggaggcacctcctggagagttcacagaaag

SEQ ID NO: 2 - DNA, 24 bp, synthetic construct
  source 1..24: mol_type = other DNA
acggaagcatctctcacagtgtct

SEQ ID NO: 3 - DNA, 24 bp, synthetic construct
  source 1..24: mol_type = other DNA
aaaaagacactgtgagagatgctt

SEQ ID NO: 4 - DNA, 24 bp, synthetic construct
  source 1..24: mol_type = other DNA
acggcccggtgataaactgccacc

SEQ ID NO: 5 - DNA, 24 bp, synthetic construct
  source 1..24: mol_type = other DNA
aaaaggtggcagtttatcaccggg

SEQ ID NO: 6 - DNA, 24 bp, synthetic construct
  source 1..24: mol_type = other DNA
acgggagcaggttccatgcctcaa

SEQ ID NO: 7 - DNA, 24 bp, synthetic construct
  source 1..24: mol_type = other DNA
aaaattgaggcatggaacctgctc

SEQ ID NO: 8 - DNA, 24 bp, synthetic construct
  source 1..24: mol_type = other DNA
acggcagatgcttggccagcttgt

SEQ ID NO: 9 - DNA, 24 bp, synthetic construct
  source 1..24: mol_type = other DNA
aaaaacaagctggccaagcatctg

SEQ ID NO: 10 - DNA, 24 bp, synthetic construct
  source 1..24: mol_type = other DNA
acggtttcagagtccacgtttggc

SEQ ID NO: 11 - DNA, 24 bp, synthetic construct
  source 1..24: mol_type = other DNA
aaaagccaaacgtggactctgaaa

SEQ ID NO: 12 - DNA, 24 bp, synthetic construct
  source 1..24: mol_type = other DNA
acgggtaacaggctgctctctgtc

SEQ ID NO: 13 - DNA, 24 bp, synthetic construct
  source 1..24: mol_type = other DNA
aaaagacagagagcagcctgttac

SEQ ID NO: 14 - DNA, 1917 bp, Mus musculus
  source 1..1917: mol_type = genomic DNA
ggtcgggcacgcaagcctccacctgcgctgcacaggcactgtgagggatgcttcaaccgccactgccacgttccggtggagcccagcgtctcctgcctggtgataagctgccacctgctctgtggcgccaccttccacatgtgcaaagagtcagagcatacactcctctgccctctggagcaggttccctgcctcaactctgagtatggctgcccgctctccatggcacgccacaagctggctaagcacctgcaggtatgccccgccagcgtggtctgctgctccatggaatggatccgctggccaaacgtggactcagaaacattccttcacgagaacatcatgaaagagacccccagtgaggagtgtttggacacagccctggccctccaggaccagaaggtactcttcagatccttgaaaatggtagagcttttcccagaaaccagagacgccacggaggaggaaccagacatgaatggtgacaccagttgggaggagactgggggcgcagtggggggagtggatgccaggctagcaccaaattcttgtttgccggcaaccagtaggcaaatgatggagctcagtcaggaggaacgagatgcactggcaaaaaccaaagaagggatggacctggacaagtttggcaagtgggaaagtatgttcagcaaggagcacgcagcctctgttttaacaggttccttggggaagagtgaagacaagaatggggatgtggctgggaaagagcagtgctccagtaatgttcgcataggggatgcagagggttctgccgaaaggagaggaccacaggaaagccagaagtcccaggaacttcccgctaccatggagatgacagggcttgctccctggcaagatggtgttctggaaagactaaaaacagccgtggatgccaaagactataacatgtatctggtgcacaacgggcggatgctcatccactttgggcagatgcctgcttgtacgcccaaggagagggactttgtttatggcaacctagaggctcaagaagtgaaaacggtttataccttcaagattcccgtgagctactgcgggaagcgagctcgcttgggagatgccatgttgaaatgcagaccaagtgaacacaaggctgtggacacttctgatctgggcatctctgtggaggacctgcccaaatctgatctcatcaagaccaccctccagtgtgctttggaaagagaactcaaaggccatgtcatctctgaatccaggagcatcgatggactctttatggaccttgccacccaaacatacaactttgagccagaacagttttcctcagagacagtgttggctgatctcctaggcactgcccaacccgggggcctgcacgtggagctccacagcgagtgtgtgactagaagacacaacaaaagcagctcagcttttacgttcacctgcaacaaattcttccgaagggatgaatttcccctgcacttcaagaatgtccacactgacatccagtcgagtctcgatggctggttccagcatcgatgccccctagcctacttgggatgtacatttgttcagaaccacttccgcccccctggacaaaaagcaaaagtgatctatagccaggagctgaagacctttgccatcaaaccagaggttgctccggagctgagtgagaaatggaagagcgaccatctctctggccgtgatgggaaaagcctgaattctctaaccagcctgcccctggaggtgttgcaatacattgctgggtttctggacagcatcagcctgtcccagctgtcccaggtgtcagtactgatgaggaacatctgtgccactttgttgcaagagagagggatggtcctctcgcaatggaagaagaagaggtattctcacggaggcacctcctggaaagtccacaatcag

SEQ ID NO: 15 - DNA, 20 bp, synthetic construct
  source 1..20: mol_type = other DNA
gactatcatatgcttaccgt

SEQ ID NO: 16 - DNA, 20 bp, synthetic construct
  source 1..20: mol_type = other DNA
tggtggttgatcatgtgaat

SEQ ID NO: 17 - DNA, 20 bp, synthetic construct
  source 1..20: mol_type = other DNA
ggcaagtcctgtcttttcta

SEQ ID NO: 18 - DNA, 20 bp, synthetic construct
  source 1..20: mol_type = other DNA
agcagtcacttgttttctct

SEQ ID NO: 19 - DNA, 20 bp, synthetic construct
  source 1..20: mol_type = other DNA
cttgacttagctctaccacc

SEQ ID NO: 20 - DNA, 17 bp, synthetic construct
  source 1..17: mol_type = other DNA
gtaaaacgacggccagt

SEQ ID NO: 21 - DNA, 17 bp, synthetic construct
  source 1..17: mol_type = other DNA
gtcatagctgtttcctg

(19) United States
(12) Patent Application Publication
(10) Pub. No.: US 2026/0248115 A1
(43) Pub. Date: Aug. 27, 2026

(54) METHOD FOR KNOCKING OUT BOVINE FBXO40 GENE AND APPLICATION THEREOF

(71) Applicants: Beijing Forestry University, Beijing (CN); Jilin Allgenes Science &Technology Co., Ltd, Songyuan City (CN)

(72) Inventors: Zhengrong Yuan, Beijing (CN); Fengcheng Song, Beijing (CN); Xichun Li, Songyuan City (CN); Guopeng Liang, Songyuan City (CN); Biao Ma, Songyuan City (CN); Wei Wang, Songyuan City (CN); Yiran Cheng, Songyuan City (CN); Shuaishuai Feng, Songyuan City (CN); Lei Yu, Songyuan City (CN); Jiaxin Cui, Songyuan City (CN)

(73) Assignees: Beijing Forestry University, Beijing (CN); Jilin Allgenes Science &Technology Co., Ltd, Songyuan City (CN)

(21) Appl. No.: 19/307,131

(22) Filed: Aug. 22, 2025

(30) Foreign Application Priority Data
Feb. 26, 2025 (CN) 202510215990.9

(51) Int. Cl. A01K 67/0276; C12N 9/22; C12N 15/11; C12N 15/90

(52) U.S. Cl. CPC A01K 67/0276; C12N 9/226; C12N 15/11; C12N 15/907; A01K 2217/054; A01K 2217/075; A01K 2227/101; C12N 2310/20

(57) ABSTRACT

The disclosure belongs to the technical field of animal bioengineering. Disclosed is a method for knocking out a bovine FBXO40 gene and an application thereof. A CRISPR/Cas12i system is used to knock out the bovine FBXO40 gene. The CRISPR/Cas12i system targets exon 5 of the bovine FBXO40 gene, and a nucleotide sequence of the exon 5 of the bovine FBXO40 gene is shown in SEQ ID No. 1. In the disclosure, a method for precise knockout of the bovine FBXO40 gene using a CRISPR/Cas12i gene editing technology is provided, and stably inheritable monoclonal cell lines and embryos with gene knockouts are successfully constructed. The method can provide experimental references for studying a molecular mechanism for bovine skeletal muscle development, and can also be applied in the breeding of new cattle breeds with high-meat-yielding.

### REFERENCE TO AN ELECTRONIC SEQUENCE LISTING

The contents of the electronic sequence listing (“USFM25080545_Sequence_Listing”, size is 23,166 bytes and it was created on Dec. 4, 2025) is herein incorporated by reference in its entirety.

### TECHNICAL FIELD

The disclosure belongs to the technical field of animal bioengineering, and specifically relates to a method for knocking out a bovine F-box only protein 40 (FBXO40) gene and an application thereof.

### BACKGROUND

In recent years, with the improvement of living standards and the upgrading of consumption structure, people increasingly demand for beef. Cultivating new breeds of beef cattle with high-meat-yielding is an important direction for the development of the beef cattle industry. Identifying key regulatory factors for the skeletal muscle development in beef cattle and elucidating the regulatory mechanisms of the skeletal muscle development in beef cattle can provide an important theoretical basis for the selection and breeding of beef cattle with high-meat-yielding and the development of biological engineering breeding materials. Skeletal muscle development in animals involves multiple molecular mechanisms, of which, insulin-like growth factor-1 (IGF1) gene plays a crucial regulatory role. IGF1 interacts with the transmembrane protein insulin-like growth factor 1 receptor (IGF1R) to induce phosphorylation of insulin receptor substrate (IRS1), thereby activating phosphatidylinositol 3-kinase/protein kinase B (PI3K/Akt) signaling pathway and regulating the proliferation and differentiation of myoblasts.

F-box protein is a key component of a Skp1-Cullin1-F-box protein (SCF) complex, which exerts E3 ubiquitin ligase activity and participates in the regulation of cell cycle and signal transduction functions. FBXO40 serves as a muscle-specific expressed gene, which has significantly enhanced expression in the skeletal muscle of patients with myasthenia gravis. Studies have shown that the FBXO40 gene targets IRS1 for ubiquitin degradation, so as to inactivate the IGF1/IRS1/PI3K/Akt signaling pathway, thereby inhibiting the development of muscle. After knocking down the FBXO40 gene in C2C12 cells (mouse myoblasts), the protein expression of IRS1 is significantly upregulated. After induced differentiation, the diameter of myotubes with the knockdown of the FBXO40 gene is increased by about 50%. Currently, there are no reports on the FBXO40 gene in cattle, including the expression pattern and biological function of the FBXO40 gene.

The clustered regularly interspaced short palindromic repeat (CRISPR)/Cas system, initially discovered in bacteria and archaea, is an immune system providing adaptivity to viruses by cleaving exogenous nucleic acids. The CRISPR/Cas system recognizes target DNA via sgRNA and mediates site-specific cleavage of the target DNA by Cas proteins, so as to induce a double-strand break (DSB). After the DSB is formed, the DNA can be repaired via non-homologous end joining (NHEJ) or homologous recombination (HR). The NHEJ frequently introduces random mutations (insertion/deletion) at the break site, resulting in changes in gene function. Therefore, designing an sgRNA on the basis of the gene sequence to be edited can achieve site-specific DNA cleavage and precise gene editing. CRISPR/Cas9 is the most commonly used gene editing technology currently, but it has a lower editing efficiency in mammals.

### SUMMARY

In response to the above prior art, the disclosure provides a method for knocking out a bovine FBXO40 gene and an application thereof, to efficiently and precisely knock out the bovine FBXO40 gene.

To realize the above objective, the disclosure employs the following technical solutions: a method for knocking out a bovine FBXO40 gene is provided, which uses a CRISPR/Cas12i system to knock out a bovine FBXO40 gene. The CRISPR/Cas12i system targets exon 5 of the bovine FBXO40 gene, and a nucleotide sequence of the exon 5 of the bovine FBXO40 gene is as shown in SEQ ID No. 1.

On the basis of the above technical solutions, the disclosure can also be improved as follows.

Furthermore, the CRISPR/Cas12i system targets nucleotides at position 178 to position 197 from a 5′ terminus in the sequence of SEQ ID No. 1.

Furthermore, the method for knocking out the bovine FBXO40 gene includes the following steps:
(1) synthesizing a sense strand and an antisense strand of a DNA sequence corresponding to crRNA, nucleotide sequences of the sense strand and the antisense strand being as shown in SEQ ID No. 6 and SEQ ID No. 7;
(2) annealing the sense strand and the antisense strand to obtain a double-stranded DNA;
(3) constructing a vector expressing a Cas12i protein, and utilizing the double-stranded DNA to construct a vector expressing the crRNA; and
(4) introducing the vector expressing the Cas12i protein and the vector expressing the crRNA into a receptor cell, and screening to obtain a monoclonal cell line with homozygous knockout of the FBXO40 gene.

Furthermore, the vector expressing the Cas12i protein is pCAG-Cas12i, and the vector expressing the crRNA is pUC19-U6-Cas12i.

Furthermore, the receptor cell is a bovine fetal fibroblast.

Furthermore, the method for knocking out the bovine FBXO40 gene is used for the preparation of a cattle breed with the FBXO40 gene knockout.

Furthermore, the preparation of the cattle breed with FBXO40 gene knockout includes the following steps: using the monoclonal cell line with homozygous knockout of the FBXO40 gene as a donor cell for nuclear transfer and an oocyte as a receptor cell for nuclear transfer, and obtaining a monoclonal embryo via a somatic cell nuclear transfer technology.

Furthermore, the method for knocking out the bovine FBXO40 gene is used for the improvement of an animal germplasm resource.

The beneficial effects of the disclosure are as follows: the CRISPR/Cas12i system is a Type V CRISPR/Cas system, which, compared with a CRISPR/Cas9 system, has the advantages of high targeting efficiency, strong specificity, smaller protein size, etc. The CRISPR/Cas12i system exhibits significant advantages, such as long knockout fragments and more PAMs, in gene editing of large agricultural animals, making it more suitable for conducting animal gene knockout experiments. The site of exon 5 of the bovine FBXO40 gene provided by the disclosure, by using the CRISPR/Cas12i gene editing system, can achieve a cleavage efficiency of 40% or more for the bovine FBXO40 gene, which can provide convenience for the preparation and production of FBXO40 gene editing materials. The FBXO40 gene editing cell line used in the disclosure is a bovine fetal fibroblast cell line, which, through a nuclear transfer technology, can be prepared into a cloned embryo, followed by transplantation to produce gene-edited live cattle. The gene editing cell lines of the disclosure are all monoclonal cell lines with homozygous knockout of the FBXO40 gene. The knocked-out fragments are short and effective, and excessively long knocked-out fragments may pose biosafety risks during the subsequent preparation of live animals. In the disclosure, all knocked-out fragments in the cell lines are less 20 base pairs (bp) in length and can form terminators, effectively halting DNA transcription and translation into proteins, thereby abolishing their biological functions.

In the disclosure, the CRISPR/Cas12i gene editing system is utilized to target a site within exon 5 of the bovine FBXO40 gene, to prepare gene editing vectors, cell lines, and embryos with precise knockout of exon 5 of the bovine FBXO40 gene, thereby achieving inactivation of the bovine FBXO40 gene function at both cellular and embryonic levels. The cell lines can be used for the preparation of cloned embryos and the study of the biological function and mechanism of the FBXO40 gene in cattle, thereby promoting the advancement of relevant scientific research. The embryos can be employed to prepare live gene-edited bovine materials and can also be applied in the breeding of new cattle breeds with enhanced meat production traits. The gene editing sites, vectors, cell lines and embryos provided by the disclosure can be applied to: (1) producing live materials for beef cattle with high-meat-yielding, (2) studying the molecular mechanism of bovine skeletal muscle development, (3) producing commercial high-quality embryo materials, and (4) exploring the action mechanism of the FBXO40 gene in animal skeletal muscle development.

### BRIEF DESCRIPTION OF THE DRAWINGS

FIG. 1 is a pCAG-Cas12i plasmid profile;

FIG. 2 is a pUC19-U6-Cas12i plasmid profile;

FIG. 3 is the mRNA expression levels of the FBXO40 gene in various tissues of cattle;

FIG. 4 is a schematic structural diagram of the bovine FBXO40 gene and a schematic diagram of a crRNA design site;

FIG. 5 is an electrophoresis gel image showing the cleavage efficiency verification of an editing vector using a T7E1 enzyme;

FIG. 6 is an electrophoresis gel image showing the cleavage efficiency verification of a pUC19-FBXO40-crRNA3 editing vector using the T7E1 enzyme;

FIG. 7 is a sequencing result image showing the cleavage efficiency verification of the pUC19-FBX040-crRNA3 editing vector through TA cloning;

FIG. 8 is a sequence diagram of a monoclonal cell line with a homozygous mutation;

FIG. 9 shows the sequencing results of a 41 #cell line;

FIG. 10 is a microscopic examination image of the 41 #cell line;

FIG. 11 shows the sequencing results of a 90 #cell line;

FIG. 12 is a microscopic examination image of the 90 #cell line;

FIG. 13 shows the sequencing results of a 140 #cell line;

FIG. 14 is a microscopic examination image of the 140 #cell line; and

FIG. 15 is a diagram showing a gene-edited cloned embryo.

### DETAILED DESCRIPTION

The specific implementations of the disclosure will be described in detail by reference to the embodiments.

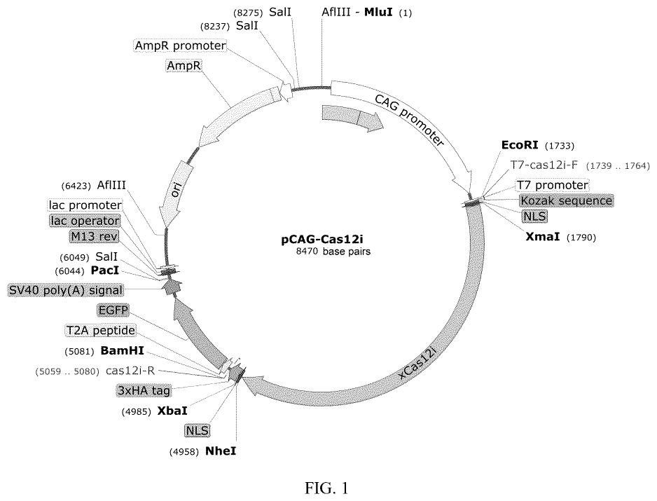

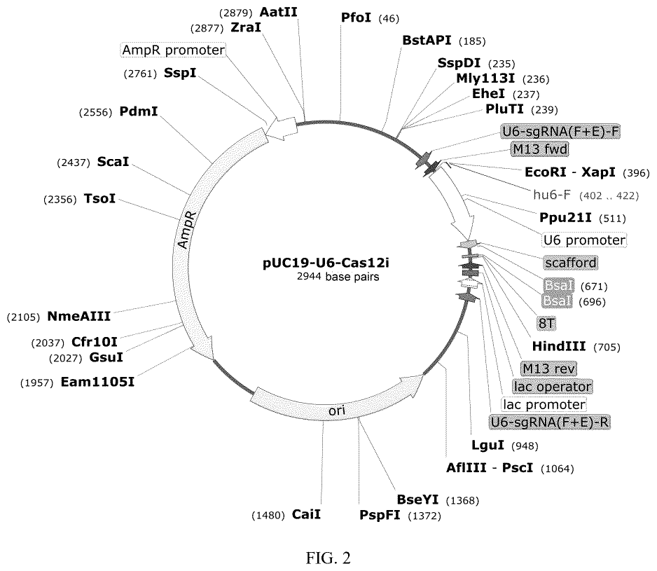

Plasmid pCAG-Cas12i (with a profile shown in FIG. 1) and plasmid pUC19-U6-Cas12i (with a profile shown in FIG. 2) refer to the patent titled “Engineered Cas12i Nuclease, Effector Protein, and Use Thereof” (Li Wei, Zhou Qi, Chen Yangcan, et al. Engineered Cas12i Nuclease, Effector Protein, and Use Thereof: 202280035920.X [P]. Application Date: 25 May 2022; Publication Date: 2 Jan. 2024). The heart, liver, spleen, lung, kidney, longissimus dorsi muscle, and subcutaneous fat of cattle are obtained from Pingliang red cattle. Unless otherwise specified, all other reagents are commercially available and purchased directly. The cell biology and molecular biology experimental methods employed are all conventional.

Ear tissues from 40-day-old Huaxi cattle fetuses are collected, and bovine fetal fibroblast cell lines are prepared following conventional method in “Cells: A Laboratory Manual” (Volume I), authored by D. L. Spector et al. (US), translated by Huang Peitang et al., published by Science Press, Beijing, February 2001; Chapter 1, Section 4, pages 27-31. Ear tissues from 6-month-old Huaxi cattle are collected, and bovine ear fibroblast cell lines are prepared using conventional methods.

### Embodiment 1

Expression pattern of the FBXO40 gene in cattle:

The bovine FBXO40 gene sequence was obtained from the National Center for Biotechnology Information (NCBI) website, with Gene ID of 613597. RNA was extracted from bovine heart, liver, spleen, lung, kidney, longissimus dorsi muscle, and subcutaneous fat using the Trizol method and reverse-transcribed into cDNA. Using B-Actin as an internal reference, real-time quantitative reverse transcription PCR (RT-qPCR) experiments were conducted to detect the mRNA expression levels of the FBXO40 gene in different tissues.

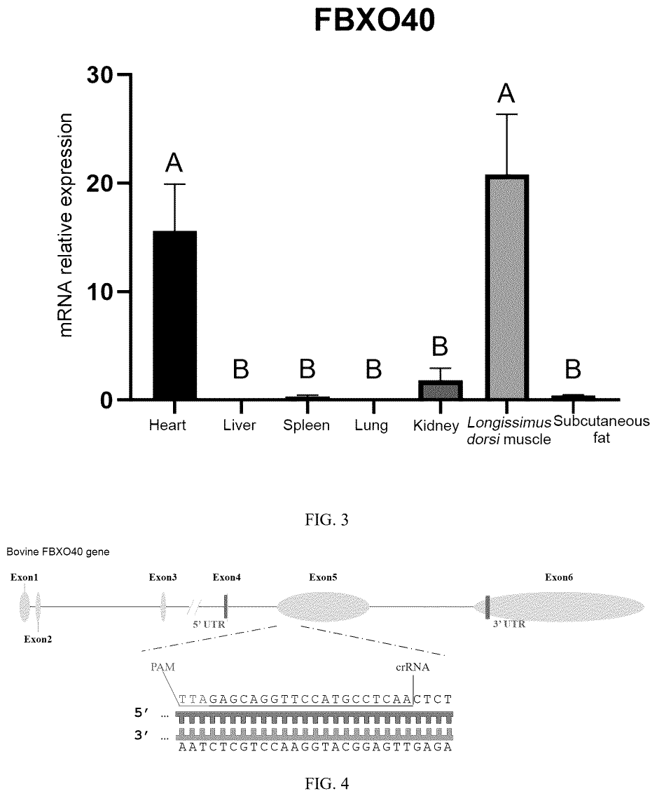

The results are shown in FIG. 3 (where A and B indicate statistically significant differences between different treatments), showing high expression of the FBXO40 gene in bovine heart tissue and longissimus dorsi muscle. The results are consistent with those found in mice and humans. Additionally, the similarity between the sequence of exon 5 of the bovine FBXO40 gene and that of mice (NCBI Gene ID: 207215, SEQ ID No. 14) reaches 81.64%.

The sequence of exon 5 of the bovine FBXO40 gene is as follows:

(SEQ ID No. 1)
5′-ggtaggacacgcaggcctccgccagggcagcacagacactgtgagagatgcttcaaccgtcactgccacatgc
ctgtggagcccagtgtctcttgcccggtgataaactgccacctgtcctgcggtgctaccttccacatgtgcaaagaggctgagcacgag
ctactctgccctttagagcaggttccatgcctcaactctgaatatggctgccccctcttcatgtcccgccacaagctggccaagcatctgc
aagtgtgtcctgccagcgtggtctgctgctccatggagtggaaccgttggccaaacgtggactctgaaacaacgcttcatgagaacatc
atgaaagagacccccgatgaggaatgcttggacacagccctggcccttcaggaccagaaggtcctttttaggtctctgaaaatggtaga
acttttcccagaaactagagaacccacggaagaggaacctcctatgaatggtgaagccagctgggaggaaatggatggagcggtgg
gtggggaggatgctggtttggtaacaggctgctctctgtcagccatgaatggggaggtggtagagctaagtcaagaagaacgagagg
cgttagccaaaaccaaagaagggatggacctggccaagtttgacaagtgggaaaatatgttcagcaaagagcatgcggcctcagcttt
aacaagctcatcagccagcagtgacagcaaaagcaggaacggcccagagaaagaacagatttccaacagcaatagaatggtagag
gagggagctgccaaaggaaaagaaccacaggaagaccagaagcagcaggactttcatgcagccatagaaaagacaggacttgccc
cttggcaggatggtgttctggaaagactgaaaacagctgtggatgcaaaggactataatatgtacctggtgcacaatgggaggatgctt
attcactttggtcagatgcctgcttgtacacccaaagagagagactttgtttacggcaacctggaggctcaggaagtgaagactgtttaca
ccttcaaagttccagtgagctactgtgggaagcgggctcgcctaggtgatgccatgagttgtaggccaagtgagcacaaagccgttgat
acttcagatttagggatcactgtggaggacctgcccaagtcagatctcatcaagaccaccctcctgtgtgctttggaaagagaactcaaa
ggtcacgtcatctccgagtccaggagcattgacgggctgttcatggatttcgctacacagacatacagttttgagccagaacagttttcttc
tgggacggtgctggccgacctcttaaccactgccaacccaaacagcaatgcgaatgggctccacgtggagctccacagtgagtgtgt
gaccaggagacacaacaagagcagctctgcctttactttcacctgcaacaaattcttcaggagggatgaatttcccctacatttcaagaat
gttcacacagacattcagtcatgtctcaacggctggttccaacaccgatgccccctggcctacttgggatgtacctttattcaaaaccattt
ccgtcccccagggcaaaaggcaaaagtgatctacagtcaggagctcaagacctttgccatcaagccggaggttgcttcagagctgagt
gagggaaggaagaacaaccatttctcaggtcgtgaaggaaaaaaccagaattctctaaccagcctgcccctggaggtcctgcagtac
attgctgggttcctagacagcatcagcctgtcccagctctcccaggtgtccgtgctgatgaggagtatctgtgccactttgttacaagaga
gagggatggtcctcctgcagtggaagaagaagaggtattcccatggaggcacctcctggagagttcacagaaag-3′.

The sequence of exon 3 of the mouse Fbxo40 gene is as follows:

(SEQ ID No. 14)
5′-ggtcgggcacgcaagcctccacctgcgctgcacaggcactgtgagggatgcttcaaccgccactgccacgttcc
ggtggagcccagcgtctcctgcctggtgataagctgccacctgctctgtggcgccaccttccacatgtgcaaagagtcagagcataca
ctcctctgccctctggagcaggttccctgcctcaactctgagtatggctgcccgctctccatggcacgccacaagctggctaagcacct
gcaggtatgccccgccagcgtggtctgctgctccatggaatggatccgctggccaaacgtggactcagaaacattccttcacgagaac
atcatgaaagagacccccagtgaggagtgtttggacacagccctggccctccaggaccagaaggtactcttcagatccttgaaaatgg
tagagcttttcccagaaaccagagacgccacggaggaggaaccagacatgaatggtgacaccagttgggaggagactgggggcgc
agtggggggagtggatgccaggctagcaccaaattcttgtttgccggcaaccagtaggcaaatgatggagctcagtcaggaggaacg
agatgcactggcaaaaaccaaagaagggatggacctggacaagtttggcaagtgggaaagtatgttcagcaaggagcacgcagcct
ctgttttaacaggttccttggggaagagtgaagacaagaatggggatgtggctgggaaagagcagtgctccagtaatgttcgcatagg
ggatgcagagggttctgccgaaaggagaggaccacaggaaagccagaagtcccaggaacttcccgctaccatggagatgacaggg
cttgctccctggcaagatggtgttctggaaagactaaaaacagccgtggatgccaaagactataacatgtatctggtgcacaacgggcg
gatgctcatccactttgggcagatgcctgcttgtacgcccaaggagagggactttgtttatggcaacctagaggctcaagaagtgaaaa
cggtttataccttcaagattcccgtgagctactgcgggaagcgagctcgcttgggagatgccatgttgaaatgcagaccaagtgaacac
aaggctgtggacacttctgatctgggcatctctgtggaggacctgcccaaatctgatctcatcaagaccaccctccagtgtgctttggaa
agagaactcaaaggccatgtcatctctgaatccaggagcatcgatggactctttatggaccttgccacccaaacatacaactttgagcca
gaacagttttcctcagagacagtgttggctgatctcctaggcactgcccaacccgggggcctgcacgtggagctccacagcgagtgtg
tgactagaagacacaacaaaagcagctcagcttttacgttcacctgcaacaaattcttccgaagggatgaatttcccctgcacttcaagaa
tgtccacactgacatccagtcgagtctcgatggctggttccagcatcgatgccccctagcctacttgggatgtacatttgttcagaaccac
ttccgcccccctggacaaaaagcaaaagtgatctatagccaggagctgaagacctttgccatcaaaccagaggttgctccggagctga
gtgagaaatggaagagcgaccatctctctggccgtgatgggaaaagcctgaattctctaaccagcctgcccctggaggtgttgcaatac
attgctgggtttctggacagcatcagcctgtcccagctgtcccaggtgtcagtactgatgaggaacatctgtgccactttgttgcaagaga
gagggatggtcctctcgcaatggaagaagaagaggtattctcacggaggcacctcctggaaagtccacaatcag-3′ .

### Embodiment 2

Construction of an FBXO40 gene editing vector pUC19-FBXO40-crRNA:

S1. A bovine FBXO40 gene is transcribed and translated into an FBXO40 protein (primarily corresponding to exon 5). Inactivation of this region leads to the loss of the specific targeting function of an SCF complex for the FBXO40 protein, blocking the FBXO40 protein-mediated ubiquitin degradation of the IRS1 protein, resulting in an elevated expression level of the IRS1 protein, thus activating the IGF1/IRS1/PI3K/Akt signaling pathway and promoting the development of bovine skeletal muscle. Therefore, the exon 5 region of the bovine FBXO40 gene was identified as a target region, as shown in FIG. 4 (In FIG. 4, Exon 1-Exon 6 represent exon 1-exon 6, PAM represents the protospacer adjacent motif in the CRISPR/Cas12i system, and 5′UTR and 3′UTR represent the 5′ untranslated region and 3′ untranslated region, respectively). Using the website (http://www.rgenome.net/cas-designer/), a total of six crRNAs with a PAM sequence of 5′-TTN-3′ were designed. The DNA sequences containing adapters corresponding to the six crRNA sequences targeting the bovine FBXO40 gene are as follows (SEQ ID Nos. 2-13):

crRNA1-F:
(SEQ ID No. 2)
5′-ACGGAAGCATCTCTCACAGTGTCT-3′;
crRNA1-R:
(SEQ ID No. 3)
5′-AAAAAGACACTGTGAGAGATGCTT-3′;
crRNA2-F:
(SEQ ID No. 4)
5′-ACGGCCCGGTGATAAACTGCCACC-3′;
crRNA2-R:
(SEQ ID No. 5)
5′-AAAAGGTGGCAGTTTATCACCGGG-3′;
crRNA3-F:
(SEQ ID No. 6)
5′-ACGGGAGCAGGTTCCATGCCTCAA-3′;
crRNA3-R:
(SEQ ID No. 7)
5′-AAAATTGAGGCATGGAACCTGCTC-3′;
crRNA4-F:
(SEQ ID No. 8)
5′-ACGGCAGATGCTTGGCCAGCTTGT-3′;
crRNA4-R:
(SEQ ID No. 9)
5′-AAAAACAAGCTGGCCAAGCATCTG-3′;
crRNA5-F:
(SEQ ID No. 10)
5′-ACGGTTTCAGAGTCCACGTTTGGC-3′;
crRNA5-R:
(SEQ ID No. 11)
5′-AAAAGCCAAACGTGGACTCTGAAA-3′;
crRNA6-F:
(SEQ ID No. 12)
5′-ACGGGTAACAGGCTGCTCTCTGTC-3′;
and
crRNA6-R:
(SEQ ID No. 13)
5′-AAAAGACAGAGAGCAGCCTGTTAC-3′.

S2. Forward and reverse sequences of the six crRNAs with adapters were synthesized into oligonucleotide chains, which were annealed. An annealing system included 2 μL of CutSmart® Buffer, 9 μL of the forward (F) sequence, and 9 μL of the reverse (R) sequence, which were reacted at 99° C. for 10 min. The original plasmid pUC19-U6-Cas12i was digested using the BsaI restriction enzyme, with a digestion system including 10 μL of CutSmart® Buffer, 10 μg of plasmid, 10 μL of BsaI enzyme, and ddH₂O added to a final volume of 100 μL, which were reacted at 37° C. for 4 h. Subsequently, the annealed crRNA was ligated to the digested plasmid, with a ligation system containing 5 μL of 2×solution I, 4 μL of the annealed product, and 1 μL of the plasmid, which were reacted at 16° C. for 3 h.

S3. The ligation product was transformed into Escherichia coli Fast-T1 competent cells, which were added to 1 mL of a liquid LB medium containing ampicillin (Amp), followed by recovery in a 37° C. shaker for 4 h. A bacterial solution was then centrifuged, and the supernatant was discarded. The cells were resuspended using 100 μL of liquid LB medium and then spread onto a solid LB medium plate containing Amp for culture overnight at 37° C.

S4. The next day, single colonies were selected and streaked onto a plate, followed by culture at 37° C. for 8 h. A portion of the bacteria from each streak was transferred into 1 mL of liquid LB medium containing Amp and cultured in a 37° C. shaker for 4 h. The bacterial solution was subjected to sequencing using the universal primer HU6-F. Bacterial colonies that had been verified by sequencing were selected to successfully construct the recombinant plasmid, and were added to 50 mL of a liquid LB medium containing Amp, followed by culture in a 37° C. shaker overnight. The next day, the recombinant plasmid pUC19-FBXO40-crRNA1-6 was extracted using a plasmid extraction kit (D6950-02) from the company OMEGA for concentration measurement, and the recombinant plasmid was stored in a refrigerator at −20° C.

The sequence of the universal primer HU6-F is as follows:

(SEQ ID NO. 15)
5′-GACTATCATATGCTTACCGT-3′.

### Embodiment 3

### Verification of the Efficiency of the FBXO40 Gene Editing Vector:

S1. Bovine ear fibroblasts were recovered and transferred into a 6-well plate, followed by culture in a complete culture medium (DMEM+10% fetal bovine serum (FBS)+1% penicillin-streptomycin double antibiotic) for 24 h. The plasmid pUC19-FBXO40-crRNA1-6 and the plasmid pCAG-Cas12i were co-transfected into the cells using Lipo 3000 liposome transfection reagent. The plasmid amounts used for transfection were 2 μg of pUC19-FBX040-crRNA1-6 and 1 μg of pCAG-Cas12i. After 48 h of culture, the cells were digested with trypsin, centrifuged to remove the supernatant, and then resuspended in 200 μL of resuspension buffer (DMEM+1% FBS). A collection buffer (DMEM+15% FBS) was prepared.

S2. The resuspended cells were sorted using a flow cytometer, and fluorescent cells were sorted into the collection buffer using a BD FACS Aria SORP flow cytometer. The cell genome was extracted from the sorted cells using a genome extraction kit, and a target region was amplified by nested PCR, with a PCR system including 1 μL of genome/template, 0.5 μL each of forward and reverse primers, 12.5 μL of KOD ONE PCR Mix, and ddH₂O added to a total volume of 50 μL.

The primer sequences for the nested PCR are as follows:

outer primer-F:
(SEQ ID No. 16)
5′-TGGTGGTTGATCATGTGAAT-3′;
outer primer-R:
(SEQ ID No. 17)
5′-GGCAAGTCCTGTCTTTTCTA-3′;
inner primer-F:
(SEQ ID No. 18)
5′-AGCAGTCACTTGTTTTCTCT-3′;
and
inner primer-R:
(SEQ ID NO. 19)
5′-CTTGACTTAGCTCTACCACC-3′.

The amplification procedure of the nested PCR: first, an outer band was amplified, and the outer band was used as a template to amplify an inner band. The amplification procedure was: 98° C. for 5 min; 98° C. for 10 s, 55° C. for 5 s, 68° C. for the outer primer for 15 s, the inner primer for 1 s, and 35 cycles; and 68° C. for 5 min, stored at 4° C.

S3. 15 μL of PCR product was taken for annealing using the Touchdown procedure: 95° C. for 10 min, then cooled from 80° C. to 20° C. at a rate of −10° C./min and stored at 4° C. The T7E1 enzyme was capable of recognizing and cleaving mismatched regions within double-stranded DNA, and these regions typically arise from error DNA repair after Cas protein cleavage. Therefore, the annealed product was subjected to T7E1 enzyme digestion to verify its cleavage efficiency. A digestion system included 15 μL of annealed products, 2.5 μL of 10×NEB buffer 2, 0.5 μL of T7E1 enzyme, and ddH₂O added to a total volume of 20 μL. The reaction conditions included incubation at 37° C. for 1 h, followed by 75° C. for 5 min. Subsequently, agarose gel electrophoresis was performed.

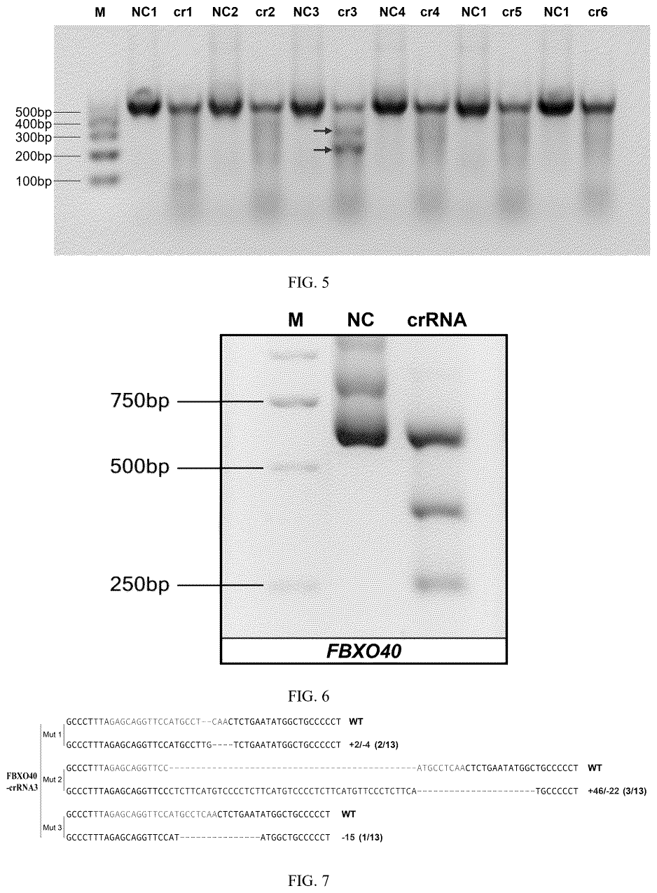

The agarose gel electrophoresis results are shown in FIG. 5. In FIG. 5, M represents the Marker, cr1-cr6 represent the treatment of the plasmid pUC19-FBXO40-crRNA1-6, and NC1-NC6 represent the blank controls corresponding to the treatment of the plasmid pUC19-FBXO40-crRNA1-6. The results show that, among the six crRNAs, crRNA3 has the most obvious cleavage effect. Therefore, pUC19-FBXO40-crRNA3 was used for subsequent experiments.

S4. pUC19-FBXO40-crRNA3 was transfected again according to the above steps (S1-S3), and the genome was extracted for PCR. After T7E1 enzymatic digestion, the agarose gel electrophoresis results are shown in FIG. 6. In FIG. 6, M represents Marker, crRNA represents the treatment of the plasmid pUC19-FBX040-crRNA3, and NC represents a blank control. ImageJ v1.54 g software was used to analyze the grayscale values of bands, so as to calculate cleavage efficiency. The calculation formula was cleavage efficiency=grayscale value of the two cleaved bands/total grayscale value of all three bands*100%. The final calculated cleavage efficiency was 47.60%.

S5. A column recovery kit was used to purify the remaining PCR products from step S4. After purification, the products were linked to the pMD19-T vector for TA cloning. After conversion and plate spreading, single clones were selected for colony PCR. 50 μL system, primers and enzyme amounts were the same as described before. A sterile pipette tip was used to select the single colonies, which were placed into the system. The amplification procedure was the same as above. The single colonies were sent to a company for sequencing using the universal primer M13.

The sequence of the universal primer M13 is as follows:

M13-F:
(SEQ ID No. 20)
5′-GTAAAACGACGGCCAGT-3′;
and
M13-R:
(SEQ ID NO. 21)
5′-GTCATAGCTGTTTCCTG-3′.

Sequencing results show 13 are successful, of which 6 undergo mutations. The calculated cleavage efficiency is 46.15%, as shown in FIG. 7, and is approximately consistent with that calculated using T7E1 enzymatic digestion, showing a high cleavage efficiency.

### Embodiment 4

Preparation of monoclonal cell line with homozygous FBXO40 gene knockout:

S1. Bovine fetal fibroblasts were recovered and transferred into a T25 culture flask, followed by culture in a complete culture medium for 48 h. The cells were digested with trypsin, centrifuged at 1000×g for 5 min, and the supernatant was discarded. The cells were resuspended using a Nucleofector electroporation solution from a Nucleofector transfection kit to obtain a cell suspension.

S2. 2 μg each of the plasmid pUC19-FBXO40-crRNA3 and the plasmid pCAG-Cas12i selected in Embodiment 3, were mixed with the cell suspension. Electroporation was performed using the U023 program. The electroporated cells were transferred to a T25 culture flask and cultured in a complete medium for 48 h.

S3. The electroporated cells were digested with trypsin, and a flow cytometer was used to sort fluorescent cells. Individual fluorescent cells were directly placed into each well of a 96-well plate pre-filled with a complete culture medium, followed by cell culture. The cells with good growth were passaged to a 48-well plate, and this process was repeated until they were passaged to a 6-well plate, with a cell confluence of 80% or more.

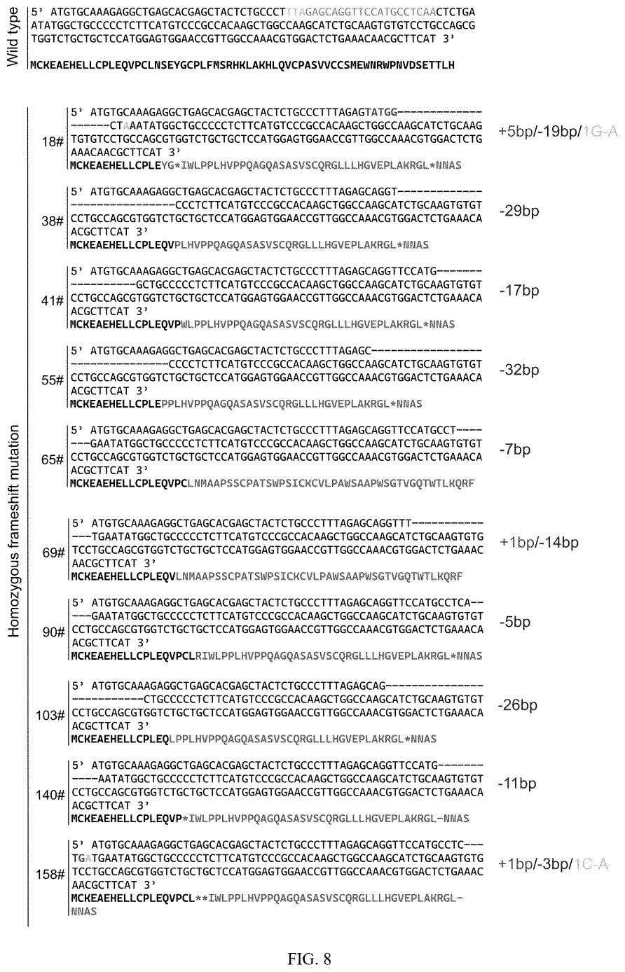

S4. The cells were digested with trypsin, and for each monoclonal cell line, a portion of the cells was added to a cryopreservation solution (DMEM+20% FBS+10% dimethyl sulfoxide) and subjected to gradient freezing before storage in a liquid nitrogen tank for later use. The remaining cells were inoculated into a 12-well plate. When the cell confluence exceeded 80%, the cell genome was extracted using a genomic DNA extraction kit, and a target region was amplified using the aforementioned nested PCR amplification method, followed by sequencing of a PCR product. The sequencing results show a single peak, and the monoclonal cell line with altered genotypes is identified as a homozygous edited monoclonal cell line. Among the 160 cultured monoclonal cell lines, a total of 14 are homozygous edited monoclonal cell lines, with 10 of them exhibiting frameshift mutations. The sequence information for these 10 frameshift mutant monoclonal cell lines is shown in FIG. 8, where #18, #38, #41, #55, #65, #69, #90, #103, #140, and #158 represent the identification numbers of the frameshift mutant monoclonal cell lines.

### Embodiment 5

Preparation of cloned embryos with homozygous FBXO40 gene knockout via nuclear transfer:

S1. A monoclonal cell line with the homozygous FBXO40 gene knockout was utilized as a donor cell for nuclear transfer. Three cell lines from Embodiment 4 were selected as donor cells for nuclear transfer.

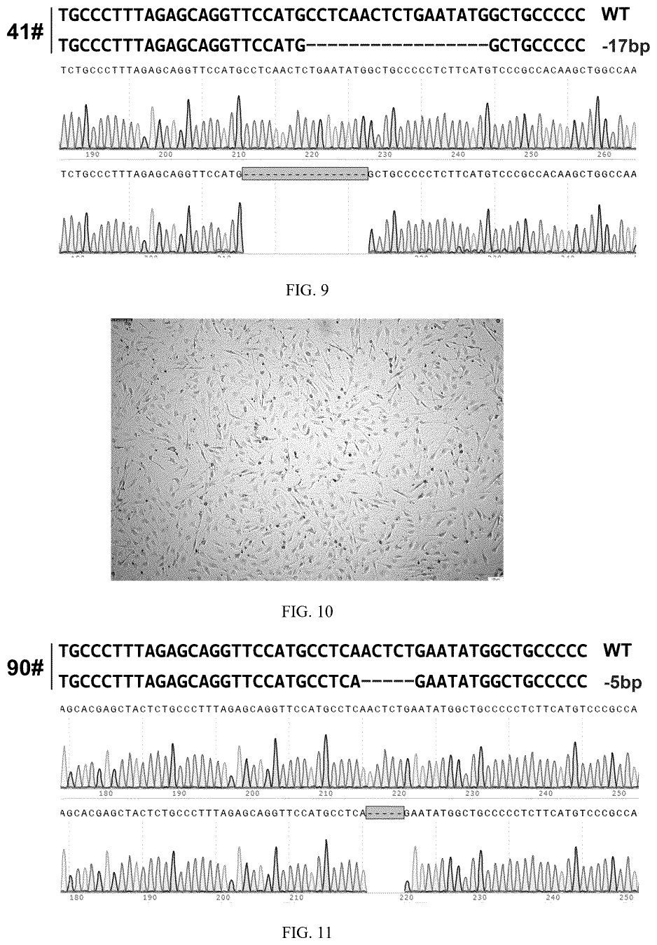

S2. The first donor cell was homozygous mutant #41 cell line, with a mutation type being the deletion of 17 bp nucleotides, spanning from position 192 to 208 at the 5′ terminus of exon 5 (SEQ ID No. 1) of the bovine FBXO40 gene, resulting in the formation of a terminator at an amino acid of position 102, thereby achieving the purpose of knocking out the gene. The sequencing results and microscopic images of the #41 cell line are shown in FIG. 9 and FIG. 10, respectively.

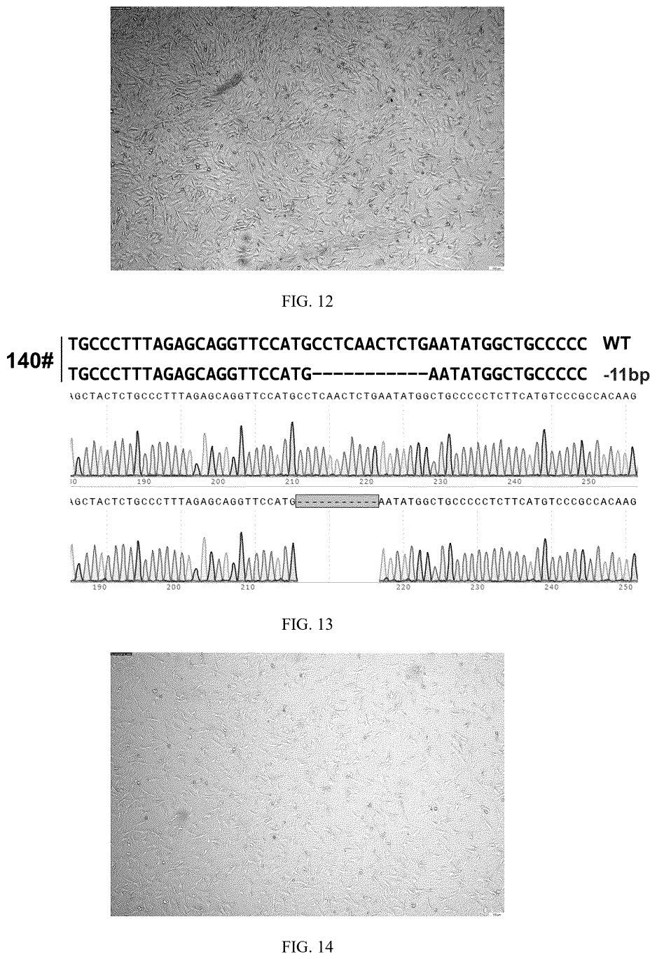

S3. The second donor cell was the homozygous mutant #90 cell line, with a mutation type being the deletion of 5 bp nucleotides, spanning from position 197 to 201 at the 5′ terminus of exon 5 (SEQ ID No. 1) of the bovine FBXO40 gene, resulting in the formation of a terminator at an amino acid of position 106, thereby knocking out the gene. The sequencing results and microscopic images of the #90 cell line are shown in FIG. 11 and FIG. 12, respectively.

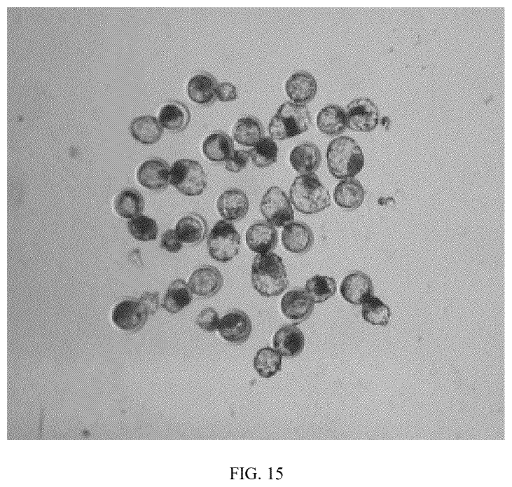

S4. The third donor cell was the homozygous mutant #140 cell line, with a mutation type being the deletion of 11 bp nucleotides, spanning from position 192 to 202 at the 5′ terminus of exon 5 (SEQ ID No. 1) of the bovine FBXO40 gene, resulting in the formation of a terminator at an amino acid of position 65, thereby achieving the purpose of knocking out the gene. The sequencing results and microscopic images of the #140 cell line are shown in FIG. 13 and FIG. 14, respectively.

All the above cell lines with relatively short deleted fragments were selected to avoid potential gene safety issues that might arise from gene deletions of large fragments.

S5. Ovaries from adult cows were obtained from a slaughterhouse, and follicles with diameters ranging from 2 to 8 mm were selected. Cumulus-oocyte complexes (COCs) with regular morphology and dense structures were recovered from these follicles. Subsequently, the COCs were placed in a four-well cell culture plate containing maturation medium (liquid M199 medium+10% FBS+0.01 U/mL bovine follicle-stimulating hormone, 0.01 U/mL bovine luteinizing hormone+1 μg/mL estradiol), with 50-60 COCs cultured per well. The plate was then placed in an incubator with 5% CO₂ at 38.5° C. for 18-20 h to allow the oocytes to mature. After that, the mature oocytes were transferred to a centrifuge tube containing 1 mg/mL hyaluronidase solution, shaken for 2-3 min, and gently pipetted with a glass pipette to achieve complete separation of cumulus cells from the oocytes. Finally, oocytes with intact morphology, uniform cytoplasmic distribution, and successful extrusion of the first polar body were selected as receptor oocytes.

S6. Oocytes with the first polar body were transferred into micromanipulation droplets (M199 medium+10% FBS+7.5 μg/mL cytochalasin B). Using a glass needle, a small incision was made in the zona pellucida above the polar body. Then, a glass pipette with an inner diameter of 20 μm was used to aspirate both the first polar body and the chromosomes beneath it from the oocyte. The oocytes, from which the first polar body and chromosomes had been removed, were then washed three times in liquid M199 medium containing 20% FBS.

S7. The aforementioned donor cells were subjected to starvation for 2-4 days and then digested. After that, uniformly shaped cells were selected and placed into the zona pellucida of the receptor oocytes through the incision. The reconstructed oocytes were placed into a Zimmerman solution (an aqueous solution containing 0.3 M mannitol, 0.1 M MgSO₄, 0.05 M CaCl₂, 0.5 mM HEPES, and 0.05 g/100 mL BSA, with a pH value adjusted to 7.2, and filtered through a 0.22 μm membrane) and equilibrated for 3-5 min. After equilibration, the reconstructed oocytes were placed in a fusion chamber, and the oocytes were rotated so that the contact surface between the donor cell and the oocyte was perpendicular to the electric field. In addition, fusion was performed using a BTX ECM-2001 fusion instrument under the following conditions: a field strength of a direct current pulse of 2.5 kv/cm, a pulse duration of 10 μs, two pulses, and a pulse interval of 1 s. After that, the reconstructed embryos were immediately transferred into a culture medium (liquid M199 medium+10% FBS) for 30 min. The reconstructed embryos were placed in an aqueous solution containing 5 μmol/L ionomycin for 4 min, then transferred to an aqueous solution containing 1.9 mmol/L 6-DMAP for 4 h, and then transferred to a CR1aa culture medium containing 5% FBS and cultured in an incubator at 38.5° C. with 5% CO₂ for 7 days. After 7 days, cloned embryos with homozygous FBXO40 gene knockout were formed (as shown in FIG. 15).

Although the specific implementation of the disclosure is described in detail by reference to the embodiments, it is to be understood that the scope of protection of the disclosure is not limited by the embodiments. All modifications and variations that can be made by a person skilled in the art without creative efforts within the scope described in the claims still fall within the scope of protection of the patent.

What is claimed is:

1. A method for knocking out a bovine FBXO40 gene, using a CRISPR/Cas12i system to knock out the bovine FBXO40 gene, wherein the CRISPR/Cas12i system targets exon 5 of the bovine FBXO40 gene, a nucleotide sequence of the exon 5 of the bovine FBXO40 gene being as shown in SEQ ID No. 1, and the CRISPR/Cas12i system targets nucleotides at position 178 to position 197 from a 5′ terminus in the sequence of SEQ ID No. 1.

2. The method for knocking out the bovine FBXO40 gene according to claim 1, comprising the following steps:
(1) synthesizing a sense strand and an antisense strand of a DNA sequence corresponding to crRNA, nucleotide sequences of the sense strand and the antisense strand being as shown in SEQ ID No. 6 and SEQ ID No. 7;
(2) annealing the sense strand and the antisense strand to obtain a double-stranded DNA;
(3) constructing a vector expressing a Cas12i protein, and utilizing the double-stranded DNA to construct a vector expressing the crRNA; and
(4) introducing the vector expressing the Cas12i protein and the vector expressing the crRNA into a receptor cell, and screening to obtain a monoclonal cell line with homozygous knockout of the FBXO40 gene.

3. The method for knocking out the bovine FBXO40 gene according to claim 2, wherein the vector expressing the Cas12i protein is pCAG-Cas12i, and the vector expressing the crRNA is pUC19-U6-Cas12i.

4. The method for knocking out the bovine FBXO40 gene according to claim 2, wherein the receptor cell is a bovine fetal fibroblast.

5. An application of the method for knocking out the bovine FBXO40 gene according to any one of claims 1-4, the method for knocking out the bovine FBXO40 gene being used in the preparation of a cattle breed with FBXO40 gene knockout.

6. The application according to claim 5, wherein the preparation of the cattle breed with FBXO40 gene knockout comprises the following steps: using the monoclonal cell line with homozygous knockout of the FBXO40 gene as a donor cell for nuclear transfer and an oocyte as a receptor cell for nuclear transfer, and obtaining a monoclonal embryo via a somatic cell nuclear transfer technology.

7. The application of the method for knocking out the bovine FBXO40 gene according to any one of claims 1-4, wherein the method for knocking out the bovine FBXO40 gene is used in the improvement of an animal germplasm resource.
